(19) United States
(12) Patent Application Publication
(10) Pub. No.: US 2026/0051037 A1
(43) Pub. Date: Feb. 19, 2026

(54) METHOD FOR MONITORING SUSPENDEDSEDIMENT CONCENTRATIONS IN MARINE DUMPING AREAS BASED ON MULTI-SOURCE REMOTE SENSING DATA

(71) Applicant: DALIAN UNIVERSITY OF TECHNOLOGY, Dalian, Liaoning (CN)

(72) Inventors: Wenyuan WANG, Dalian, Liaoning (CN); Yun PENG, Dalian, Liaoning (CN); Xinglu XU, Dalian, Liaoning (CN); Zijian GUO, Dalian, Liaoning (CN)

(21) Appl. No.: 19/191,620

(22) Filed: Apr. 28, 2025

Related U.S. Application Data
(63) Continuation of application No. PCT/CN2024/117033, filed on Sep. 5, 2024.

(30) Foreign Application Priority Data
Mar. 22, 2024 (CN) 202410331238.6

(51) Int. Cl. G06T 7/00; G01N 33/18

(52) U.S. Cl. CPC G06T 7/0002; G01N 33/18; G06T 2207/10032; G06T 2207/20221; G06T 2207/30181

(57) ABSTRACT

A method for monitoring suspended sediment concentrations in marine dumping areas based on multi-source remote sensing data is provided, which belongs to the technical field of remote sensing monitoring for suspended sediment concentrations in marine dredging and dumping areas. The method comprises: selecting a sea area comprising dumping area as research area, and acquiring measured suspended sediment concentration data. Acquiring measured suspended sediment data respectively and preprocessing remote sensing images. Matching the acquired data to form a data set, and constructing an inversion model of the suspended sediment concentrations to obtain spatiotemporal distribution images of the suspended sediment concentrations in the water body of the research area. Constructing a multi-source remote sensing spatiotemporal data fusion model to obtain a spatiotemporal distribution map of the suspended sediment concentrations with high spatiotemporal resolution in the dumping area, and monitoring the change condition of the suspended sediment concentrations in a marine dumping area.

### TECHNICAL FIELD

The present invention belongs to the technical field of remote sensing monitoring for suspended sediment concentrations in marine dredging and dumping areas, and relates to remote sensing satellite image processing, measured data of suspended sediment concentrations in monitoring stations, remote sensing reflectivity data, inversion methods for suspended sediment concentrations and spatiotemporal fusion algorithms for remote sensing images.

### BACKGROUND

The monitoring of water quality change in marine dumping areas is an important basis and foundation for understanding the use status of the dumping areas and subsequent evaluation, wherein the monitoring of suspended sediment concentrations is an important content of water quality monitoring. In the traditional monitoring methods, a specific monitoring station is used for extracting water samples and bringing them back to a laboratory to determine the concentration value of suspended sediment so as to control the intensity of marine dumping activities, but the monitoring method is low in efficiency and high in cost. Moreover, limited data cannot comprehensively summarize the sea area conditions [Yu Chuanyang, Wang Lin, Zhao Sufang, et al. Remote sensing monitoring for suspended sediment concentrations in adjacent sea areas based on multi-source satellite images [J]. Environmental Impact Assessment, 2023, 45 (5): 17-21+52]. At present, the inversion monitoring for suspended sediment by using the satellite remote sensing technology has the advantages of short monitoring period, high spatiotemporal resolution, wide observation range, etc., which improves the situation of time consumption and labor consumption in human monitoring. The current inversion methods for the suspended sediment concentrations mainly include the analytical method, the semi-analytical method, the empirical method, the machine learning method, etc., wherein the empirical method has a simple construction process, convenient use and wide application, and has been well applied in many sea areas.

However, in a small-range area with complex water quality change in a short term, such as dumping areas, higher requirements are proposed for the spatiotemporal resolution of remote sensing monitoring, while general satellite sensors are difficult to take into account both high temporal and high spatial resolutions. At present, with respect to this problem, a method for spatiotemporal fusion of multi-source remote sensing data is mainly used to solve the problem of contradiction between the temporal resolution and the spatial resolution of single remote sensing data [WU M, WU C, HUANG W, et al. An improved high spatial and temporal data fusion approach for combining Landsat and MODIS data to generate daily synthetic Landsat imagery [J]. Information Fusion, 2016, 31:14-25.]. This method mainly fuses high temporal and low spatial resolutions with the low temporal and high spatial remote sensing data through appropriate algorithms to generate high temporal and high spatial remote sensing images for better observation. At present, this method has been widely used in the fields of numerical estimation, change monitoring, ground object classification, etc. However, the research on the spatiotemporal fusion monitoring for the suspended sediment concentrations in the marine dumping areas has not been found. The present invention proposes a technical solution for the monitoring for the suspended sediment concentrations in this area.

The present invention uses a remote sensing inversion monitoring technology to perform spatiotemporal data fusion based on multi-source satellite images, solves the shortcomings of the traditional monitoring methods to a certain extent, and provides auxiliary assistance for monitoring and management of the marine dumping areas, selection, planning and use of the dumping areas, supervision of illegal dumping and other work.

### SUMMARY

In view of the problem that the existing remote sensing monitoring technology is limited by the difficulty in giving consideration to the spatiotemporal resolution of satellite sensors and cannot effectively monitor a small-range area with complex water quality change in a short term, such as the dumping areas, the spatiotemporal fusion algorithm is used to generate a spatiotemporal distribution map of the suspended sediment concentrations in temporary marine dumping areas with higher spatiotemporal resolution, so as to realize hourly dynamic monitoring for the suspended sediment concentrations in the temporary marine dumping areas and analyze the spatiotemporal change characteristics of suspended sediment generated by dredging and dumping operation.

To achieve the above purpose, the present invention adopts the following technical solution:
A method for monitoring suspended sediment concentrations in marine dumping areas based on multi-source remote sensing data fusion is provided. The monitoring method comprises: step 1: selecting a sea area comprising a dumping area as a research area, and acquiring more than 40 groups of measured suspended sediment concentration data at different places in the research area. Step 2: acquiring one satellite image with high spatial resolution (less than 50 meters) and a plurality of satellite images with high temporal resolution (less than 1 hour), which are consistent with the acquisition time of the measured suspended sediment data and cover the research area, respectively, and preprocessing remote sensing images to obtain remote sensing reflectivity. Step 3: matching the data acquired in steps 1 and 2 according to time and places to form a data set. Step 4: constructing a suspended sediment concentration inversion model based on the data set acquired in step 3, and inputting the remote sensing images obtained in step 2 into the suspended sediment concentration inversion model to obtain spatiotemporal distribution images of suspended sediment concentrations in a water body of the research area, comprising one suspended sediment concentration image with high spatial resolution (less than 50 meters) and a plurality of suspended sediment concentration images with high temporal resolution (less than 1 hour). Step 5: constructing a multi-source remote sensing spatiotemporal data fusion model through the suspended sediment concentration images acquired in step 4, and inputting the spatiotemporal distribution images of the suspended sediment concentrations with high temporal resolution (less than 1 hour) into the fusion model to obtain a spatiotemporal distribution map of the suspended sediment concentrations in the dumping area with high spatiotemporal resolution. Step 6: monitoring the change condition of the suspended sediment concentrations in a marine dumping area according to the spatiotemporal distribution map of the suspended sediment concentrations in the dumping area generated in step 5.

### Specific Steps Are as Follows:

Step 1: selecting a sea area comprising a dumping area as a research area, and acquiring more than 40 groups of measured suspended sediment concentration data at different places in the research area.
The measured suspended sediment concentration data is acquired through a water quality monitoring station in the research area.
Step 2: acquiring one satellite image with high spatial resolution (less than 50 meters) and a plurality of satellite images with high temporal resolution (less than 1 hour), which are consistent with the acquisition time of the measured suspended sediment data and cover the research area, respectively, and preprocessing remote sensing images to obtain remote sensing reflectivity.
Further, the remote sensing images come from satellites with different spatiotemporal resolutions, such as GOCI and Landsat 8, and are available for free on websites. The remote sensing images are divided into one remote sensing image with low temporal and high spatial resolutions and a plurality of remote sensing images with high temporal and low spatial resolutions. Wherein the remote sensing images with high temporal and low spatial resolutions comprise a plurality of remote sensing images with a temporal resolution of less than or equal to 1 hour on the day of the dumping operation in the dumping area.
Further, the preprocessing process of the satellite image data is: radiometric calibration, geometric correction, atmospheric correction, image cropping, and final acquisition of the remote sensing reflectivity data of the satellite images in different bands at the position of the water quality monitoring station.
Step 3: matching the data acquired in steps 1 and 2 according to time and places to form a data set, that is, matching the water body sampling time of the corresponding measured water quality station and the longitude and the latitude of the water quality station with the shooting time and longitude and latitude in the satellite images to form a data set of the suspended sediment concentrations and the remote sensing reflectivity.
Step 4: according to the data set obtained in step 3, inputting the remote sensing images obtained in step 2 by using an inversion method for the suspended sediment concentrations to obtain the spatiotemporal distribution images of the suspended sediment concentrations in the water body for each remote sensing image, wherein the spatiotemporal distribution image of the suspended sediment concentrations in the water body obtained from a remote sensing image with low temporal and high spatial resolutions is set as F, and the spatiotemporal distribution image of the suspended sediment concentrations in the water body obtained from a plurality of remote sensing images with high temporal and low spatial resolutions is set as Cᵢ; i is an integer from 1 to n, and n is the number of the remote sensing images with high temporal and low spatial resolutions; and F is set as G after resampled to the same resolution as C₁.

### Specifically:

A remote sensing inversion model of the suspended sediment concentrations in the water body of the research area is constructed based on the inversion method for the suspended sediment concentrations, and inversion is conducted to obtain the spatiotemporal distribution images of the suspended sediment concentrations in the water body for each remote sensing image in the research area, wherein

The remote sensing inversion model of the suspended sediment concentrations is an exponential model:

$$SPM = {ae}^{bx}$$

where SPM is the concentration of surface suspended sediment (g/m³), a and b are fitting coefficients, and x is a ratio of a red band value to a green band value in the remote sensing reflectivity of the data set obtained in step 3.

Step 5: constructing a multi-source remote sensing spatiotemporal data fusion model through the spatiotemporal distribution images of the suspended sediment concentrations acquired in step 4. The patent adopts an FSDA spatiotemporal data fusion model. The algorithm of the FSDAF model was proposed by Zhu, et al. [ZHU X, HELMER E H, GAO F, et al. A flexible spatiotemporal method for fusing satellite images with different resolutions [J]. Remote Sensing of Environment, 2016, 172:165-177] in 2016. The input data of the algorithm are an image pair with low and high spatial resolutions acquired from different satellite images at a certain moment and the image data with low spatial resolution at a predicting moment, and the output data are high resolution remote sensing images at the predicting moment. The patent makes some modifications to the algorithm. The image pair of the input data comprises F obtained in step 4 and G obtained by resampling F. The input image data with low spatial resolution at another predicting moment is Cᵢ; and i is an integer from 1 to n. The output data is the spatiotemporal distribution image Pᵢ of the suspended sediment concentration with high temporal and high spatial resolutions at the moment corresponding to Cᵢ, and i is an integer from 1 to n.

Step 6: monitoring the change condition of the suspended sediment concentrations in the marine dumping area according to the spatiotemporal distribution map Pᵢ of the suspended sediment concentrations with high spatiotemporal resolution generated in step 5, wherein i is an integer from 1 to n, and supervising the dumping and other work to provide auxiliary assistance to assist a management department in the supervision work. Specifically, the suspended sediment concentration of the dumping area is analyzed in the spatiotemporal distribution image of the suspended sediment concentrations, and the change trend of the concentrations is analyzed.

The present invention has the following effects and benefits:
(1) The remote sensing technology adopted by the present invention has the advantages of short monitoring period, high spatiotemporal resolution, wide observation range, etc., can synchronously monitor a wide range of sea area, has good instantaneity, and overcomes the shortcomings of the traditional monitoring methods. The spatiotemporal contradiction problem of the remote sensing images is solved by constructing the multi-source remote sensing spatiotemporal data fusion model so that the monitoring of the suspended sediment concentrations in the temporary marine dumping areas becomes more precise.
(2) The present invention can provide auxiliary assistance for supervision and management of marine dumping, monitoring and management of the marine dumping areas, selection, planning and use of the dumping areas, supervision of illegal dumping and other work, and can also assist the management department in the supervision work.

### DESCRIPTION OF THE DRAWINGS

FIG. 1 is a flow chart of a method of the present invention.

FIG. 2 is a schematic diagram of positions of a research area and a dumping area.

FIG. 3 is a spatiotemporal distribution map of suspended sediment concentrations in a dumping area; FIG. 3 (a) is a spatiotemporal distribution map of the suspended sediment concentrations in the dumping area at 8:15; FIG. 3 (b) is a spatiotemporal distribution map of the suspended sediment concentrations in the dumping area at 9:15; FIG. 3 (c) is a spatiotemporal distribution map of the suspended sediment concentrations in the dumping area at 10:15; FIG. 3 (d) is a spatiotemporal distribution map of the suspended sediment concentrations in the dumping area at 11:15; FIG. 3 (e) is a spatiotemporal distribution map of the suspended sediment concentrations in the dumping area at 12:15; FIG. 3 (f) is a spatiotemporal distribution map of the suspended sediment concentrations in the dumping area at 13:15.

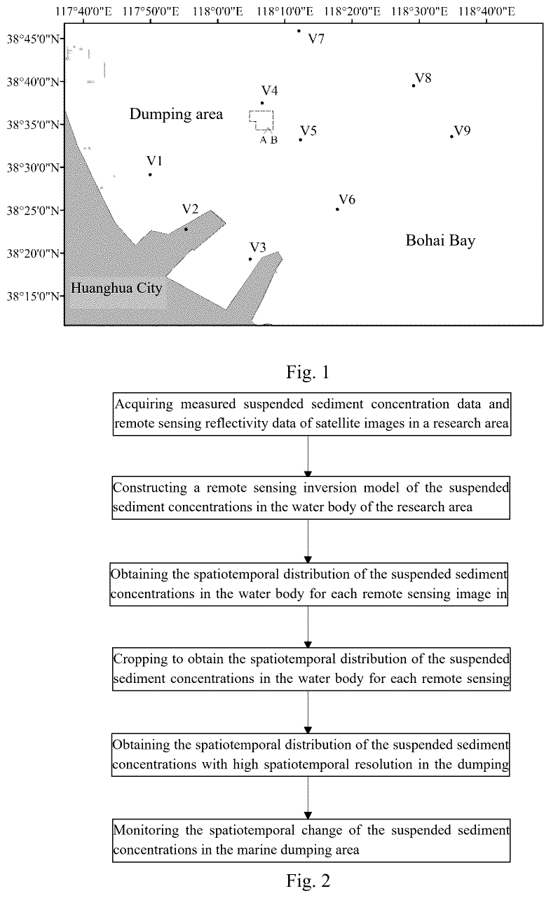

### DETAILED DESCRIPTION

The present invention will be further described below in combination with the drawings.

Taking the remote sensing monitoring of the suspended sediment concentrations in the temporary marine dumping area of dredging sediment in Huanghua port area as an example, the implementation steps of the method are divided into the following four steps.

Step 1: determining a research area, and acquiring the position information of a monitoring station and the data information of the suspended sediment monitored.

In this example, the temporary marine dumping area of Huanghua port is selected as the research area. The research area is shown in FIG. 1. V1 to V9 are nine water quality stations. Water quality samples are obtained by a ship. The surface water bodies are sampled and taken back to a laboratory to measure the concentrations of the suspended sediment. A total of 41 groups of water samples are sampled and the concentrations of the suspended sediment are measured. The dumping area is an area enclosed by the dotted line in FIG. 1.

Step 2: selecting appropriate satellite images according to the research area and conducting data preprocessing.

The satellite remote sensing data used in the research are the Geostationary Ocean Color Imager II (GOCI II) of Korea and Landsat 8, the eighth satellite of the Landsat Program of the United States. Sixteen GOCI II remote sensing images and one Landsat 8 remote sensing image are selected according to the site water quality sampling time herein, wherein the GOCI II remote sensing images come from Korea Ocean Satellite Center (https://www.nosc.go.kr/) and the Landsat 8 remote sensing images come from the EarthExplorer of the United States (Https://earthexplorer.usgs.gov). The remote sensing reflectivity is obtained after radiometric calibration, atmospheric correction and geometric correction of the images by ENVI software. The remote sensing reflectivity data is matched with the corresponding water suspended sediment concentration data to obtain 41 pieces of data of the corresponding measured stations and remote sensing reflectivity.

Step 3: inverting the concentrations of the suspended sediment according to the measured data and the remote sensing reflectivity.

The 41 pieces of data of the corresponding measured stations and remote sensing reflectivity obtained above are randomly divided into 31 groups and 10 groups. The former is used as a training set for model construction, and the latter is used for verifying the inversion model. Through Pearson correlation analysis, the correlation strength of different bands or band combinations and the suspended sediment concentrations can be obtained. A correlation coefficient between the remote sensing reflectivity and the suspended sediment concentration of each band can be obtained from the training set. The results show that bands 555, 620, 660, 680 and 709 present significant correlation with SPM. Therefore, the five bands can be used for establishing the inversion algorithm of SPM. The correlation coefficients of the five bands and the band ratios are shown in Table 1.

TABLE 1
Pearson Correlation Coefficients of Each Band and Band Combination

| | Pearson | | Number | | Pearson | | Number |
|---|---|---|---|---|---|---|---|
| Bands/Band | Correlation | | of | Bands/Band | Correlation | | of |
| Combinations | Coefficients | p | Samples | Combinations | Coefficients | p | Samples |
| 555 | 0.664 | <0.001 | 31 | 660 /709 | −0.733 | <0.001 | 31 |
| 620 | 0.805 | <0.001 | 31 | 680 / 709 | −0.707 | <0.001 | 31 |
| 660 | 0.834 | <0.001 | 31 | 620 / 555 | 0.844 | <0.001 | 31 |
| 680 | 0.843 | <0.001 | 31 | 660 /555 | 0.864 | <0.001 | 31 |
| 709 | 0.846 | <0.001 | 31 | 680 /555 | 0.870 | <0.001 | 31 |
| 555/620 | −0.760 | <0.001 | 31 | 709 /555 | 0.863 | <0.001 | 31 |
| 555 / 660 | −0.754 | <0.001 | 31 | 620 / 660 | 0.843 | <0.001 | 31 |
| 555 / 680 | −0.770 | <0.001 | 31 | 680 /620 | 0.831 | <0.001 | 31 |
| 555 / 709 | −0.726 | <0.001 | 31 | 709 /620 | 0.826 | <0.001 | 31 |
| 620 / 660 | −0.812 | <0.001 | 31 | 680 /660 | −0.069 | 0.711 | 31 |
| 620 / 680 | −0.797 | <0.001 | 31 | 709 / 660 | 0.782 | <0.001 | 31 |
| 620 / 709 | −0.755 | <0.001 | 31 | 709 /680 | 0.761 | <0.001 | 31 |
| 660 / 680 | 0.056 | 0.766 | 31 | | | | |

It can be seen that the SPM concentration is most significantly correlated with 680/555 and 660/555. The ratio bands 680/555 and 660/555 are used for establishing an exponential model for simulation analysis respectively, and the model equation, the determination coefficient R² and the root mean square error (RMSE) are calculated, as shown in Table 2.

TABLE 2
Models and Expressions of Band/Band Ratio

| Band/Band | | | | |
|---|---|---|---|---|
| Ratio | Models | Expressions | R² | RMSE |
| 660/555 | Exponential | y = 2.7542e^{3.2132x} | 0.79 | 9.87 |
| 680/555 | Exponential | y = 2.8576e^{3.2793x} | 0.77 | 10.16 |

The correlation coefficient of the 660/555 nm band is the best, and the absolute error is minimal. The corresponding ratio band exponential and the polynomial model have the best effects. Test sets are used for verification respectively.

In this example, the root mean square error (RMSE) and a mean absolute percentage error (RE) are used for assessing the inversion accuracy of the model.

Calculation formulas are as follows:

$$R M S E = \sqrt{\frac{1}{n} {\sum }_{i = 1}^{n} {( {x}_{mod , i} - {x}_{obs , i} )}^{2}}$$

$$RE = \frac{1}{n} {\sum }_{i = 1} \vert \frac{{x}_{mod , i} - {x}_{obs , i}}{{x}_{obs , i}} \vert$$

where: X_{mod,i} is a calculated value of the ith measured station; x_{obs,i} is a measured value of the ith measured station; and n represents the number of the measured stations. When 10 groups of data of a verification set are substituted into the model for verification, the RMSE is 4.38 and the RE is 18.95%. The RE is less than 20%. The inversion performance is good, and the inversion application of the suspended sediment concentrations can be conducted better. Therefore, the inversion formula obtained is:

$$SPM = 2.7542 {e}^{3.2 \; 132 x}$$

where SPM is the concentration of surface suspended sediment (g/m³), and x is a band ratio, that is, a ratio of the red band with the wavelength of 660 to the green band with the wavelength of 555. The remote sensing images preprocessed in step 2 are inputted into the inversion model for inversion to obtain the spatiotemporal distribution images of the suspended sediment concentrations in the water body in each remote sensing image of the research area, and the area in which the dumping area is reserved is cropped, wherein the spatiotemporal distribution image of the suspended sediment concentrations obtained by image inversion of Landsat 8 is set as F, resampled into a 250 m resolution and saved as G. Six of the spatiotemporal distribution images of the suspended sediment concentrations obtained from GOCI II image inversion, with time of 8:15 to 13:15 on Nov. 18, 2023, are denoted as Cᵢ, and i belongs to an integer from 1 to 8.

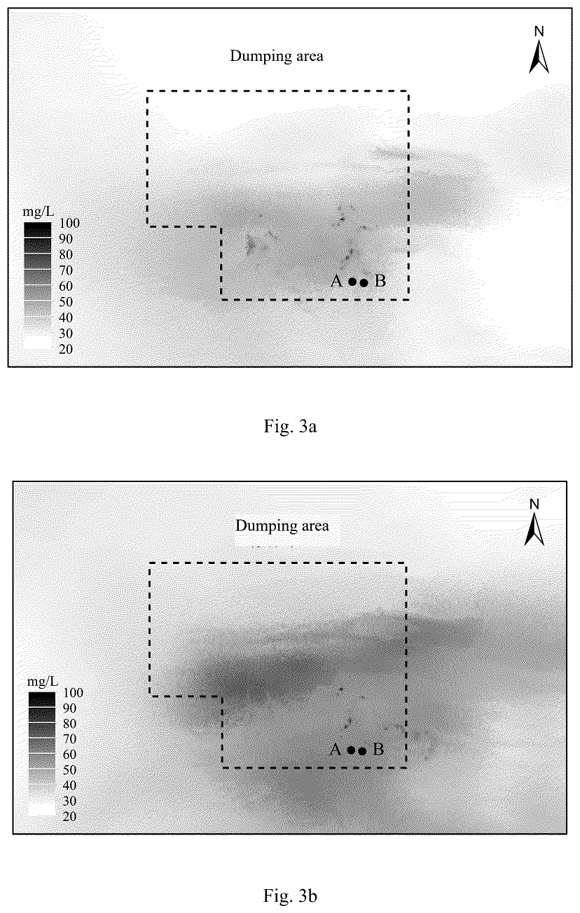

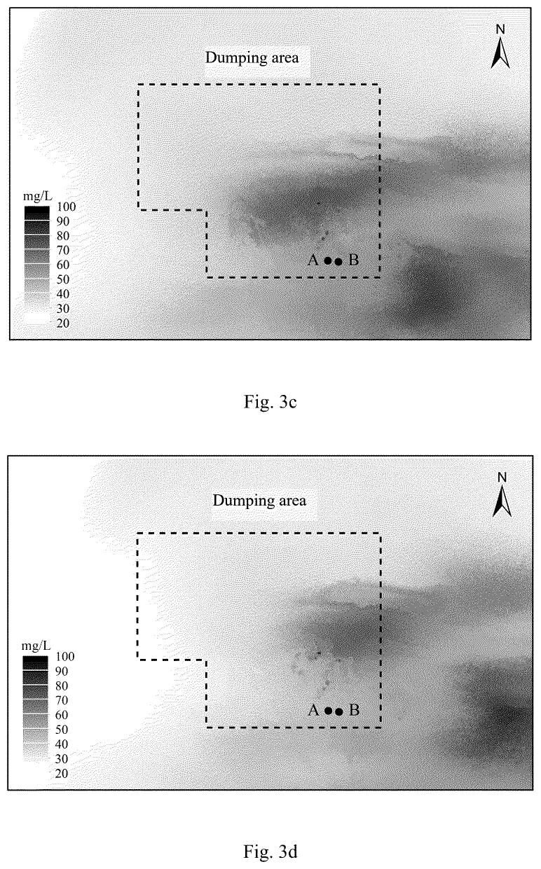

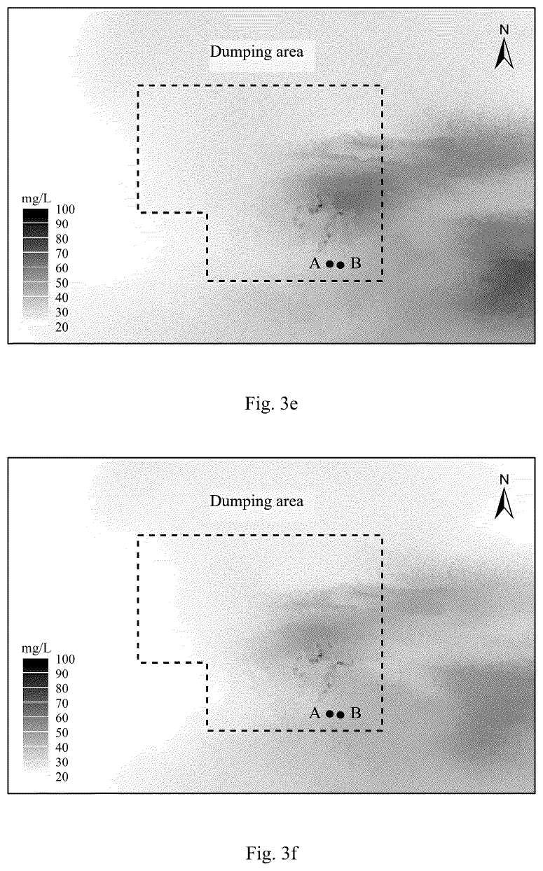

Step 4: inputting F and G of step 3 to construct an FSDAF spatiotemporal fusion model, and inputting Ci obtained by inversion of GOCI II, wherein i belongs to an integer from 1 to 8, thereby generating the spatiotemporal distribution maps of the suspended sediment concentrations in the dumping area with high spatiotemporal resolution at Beijing time of 8:15 to 13:15 on Nov. 18, 2023, as shown in FIG. 3, wherein points A and B are dumping points for dredging vessels to conduct dumping operation in the dumping area.

Step 5: monitoring the change condition of the suspended sediment concentrations in the marine dumping area according to the spatiotemporal distribution images of the suspended sediment concentrations with high spatiotemporal resolution in the dumping area generated in step 4, and monitoring and analyzing the dumping. There are areas with high concentration value of suspended lumpy sediment near the dumping area, which are obviously different from the surrounding background water bodies. The lumpy water bodies diffuse and move around with the time and the influence of the water flow, which is consistent with the diffusion movement of dredging sediment after entering the water. It can be seen from FIG. 3a that after the New RV Sirenian dredging vessel conducts the dumping operation at point A in the dumping area at 6:30 a.m., the average suspended sediment concentration in the dumping area at 8:15 am is 36.92 mg/L and the minimum value is 22.65 mg/L after 1 hour and 45 minutes of water body dilution, with little difference from the surrounding background water body. FIG. 3b shows that after the New RV Sirenian dredging vessel conducts the dumping operation at point B in the dumping area at 9:06 a.m., the average suspended sediment concentration in the dumping area is increased significantly. The average concentration is 50.27 mg/L, and the minimum value in the area is 30.92 mg/L, with obvious differences from the surrounding background water body. After the dumping operation is stopped, it can be seen from FIGS. 3c-f that the average suspended sediment concentration in the area is gradually decreased from 45.85 mg/L to 31.68 mg/L, and the rate of decrease is 4.72 mg/L per hour. The area with high concentration value of the suspended lumpy sediment diffuses to the southwest with the water flow, and the square measure of the area is gradually decreased. Until 13:15, the minimum concentration value of the suspended sediment in the dumping area is decreased to 20.34 mg/L, with little difference from the water body of the surrounding sea area. To sum up, the dumping operation in the dumping area can directly affect the concentration sizes of the suspended sediment in the area and the surrounding sea area. The water quality of the small-range sea area can be better monitored based on the hourly suspended sediment images of FSDAF inversion, and the short-term change in the sea area caused by dredging and dumping activities is further analyzed.

The above embodiments only express the implementation of the present invention, and shall not be interpreted as a limitation to the scope of the patent for the present invention. It should be noted that, for those skilled in the art, several variations and improvements can also be made without departing from the concept of the present invention, all of which belong to the protection scope of the present invention.

1. A method for monitoring suspended sediment concentrations in marine dumping areas based on multi-source remote sensing data, comprising:
step 1: selecting a sea area comprising a dumping area as a research area, and acquiring more than 40 groups of measured suspended sediment concentration data at different places in the research area through a water quality monitoring station in the research area;
step 2: acquiring one satellite image with high spatial resolution and a plurality of satellite images with high temporal resolution, which are consistent with the acquisition time of the measured suspended sediment data and cover the research area, respectively, and preprocessing remote sensing images to obtain remote sensing reflectivity;
step 3: matching the data acquired in steps 1 and 2 according to time and places to form a data set; specifically: matching the water body sampling time of the corresponding measured water quality station and the longitude and the latitude of the water quality station with the shooting time and longitude and latitude in the satellite images to form a data set of the suspended sediment concentrations and the remote sensing reflectivity;
step 4: constructing a suspended sediment concentration inversion model based on the data set acquired in step 3, and inputting the remote sensing images obtained in step 2 into the suspended sediment concentration inversion model to obtain spatiotemporal distribution images of suspended sediment concentrations in a water body for each corresponding remote sensing image of the research area, comprising one suspended sediment concentration image with high spatial resolution and a plurality of suspended sediment concentration images with high temporal resolution;
step 5: constructing a multi-source remote sensing spatiotemporal data fusion model through the suspended sediment concentration images acquired in step 4, and inputting the spatiotemporal distribution images of the suspended sediment concentrations with high temporal resolution into the fusion model to obtain a spatiotemporal distribution map of the suspended sediment concentrations in the dumping area with high spatiotemporal resolution;
step 6: monitoring the change condition of the suspended sediment concentrations in a marine dumping area according to the spatiotemporal distribution map of the suspended sediment concentrations in the dumping area generated in step 5.

2. The method for monitoring suspended sediment concentrations in marine dumping areas based on multi-source remote sensing data according to claim 1, wherein in the step 2, the preprocessing process of the satellite image data is: radiometric calibration, geometric correction, atmospheric correction, image cropping, and final acquisition of the remote sensing reflectivity data of the satellite images in different bands at the position of the water quality monitoring station.

3. The method for monitoring suspended sediment concentrations in marine dumping areas based on multi-source remote sensing data according to claim 1, wherein in the spatiotemporal distribution images of the suspended sediment concentrations in the water body in the step 4: the spatiotemporal distribution image of the suspended sediment concentrations in the water body obtained from a remote sensing image with low temporal and high spatial resolutions is set as F, and the spatiotemporal distribution image of the suspended sediment concentrations in the water body obtained from a plurality of remote sensing images with high temporal and low spatial resolutions is set as Cᵢ; i is an integer from 1 to n, and n is the number of the remote sensing images with high temporal and low spatial resolutions; and F is set as G after resampled to the same resolution as C₁; the specific process of step 4 is as follows:
constructing a remote sensing inversion model of the suspended sediment concentrations in the water body of the research area, and conducting inversion to obtain the spatiotemporal distribution images of the suspended sediment concentrations in the water body for each remote sensing image in the research area, wherein the remote sensing inversion model of the suspended sediment concentrations is an exponential model:
$$SPM = {ae}^{bx}$$
where SPM is the concentration of surface suspended sediment (g/m³), a and b are fitting coefficients, and x is a ratio of a red band value to a green band value in the remote sensing reflectivity of the data set obtained in step 3.

4. The method for monitoring suspended sediment concentrations in marine dumping areas based on multi-source remote sensing data according to claim 1, wherein the step 5 is specifically: an FSDA spatiotemporal data fusion model is used; the image pair of input data comprises F obtained in step 4 and G obtained by resampling F; the input image data with low spatial resolution at another predicting moment is Cᵢ; i is an integer from 1 to n; output data is the spatiotemporal distribution image Pᵢ of the suspended sediment concentration with high temporal and high spatial resolutions at the moment corresponding to Cᵢ; and i is an integer from 1 to n.
